(19) United States
(12) Patent Application Publication
(10) Pub. No.: US 2014/0275875 A1
(43) Pub. Date: Sep. 18, 2014

(54) SYSTEM AND METHOD FOR POSITIONING A SENSOR

(71) Applicant: Covidien LP, Mansfield, MA (US)

(72) Inventors: Mark Su, Boulder, CO (US); Friso Schlottau, Lyons, CO (US)

(73) Assignee: Covidien LP, Mansfield, MA (US)

(21) Appl. No.: 13/836,262

(22) Filed: Mar. 15, 2013

(51) Int. Cl. A61B 5/00; A61B 5/145; A61B 5/1455

(52) U.S. Cl. CPC A61B 5/684; A61B 5/14552; A61B 5/14542
USPC 600/323; 600/372; 600/383; 600/364

(57) ABSTRACT

Sensor designs or shapes to facilitate the placement of sensors on a patient are provided. For example, a first sensor may include a sensor body having a keyed interface region that is configured to align with a complementary keyed interface region of second sensor. Such sensors may also include various features to further facilitate the positioning of the sensors on the patient tissue and the positioning of the sensors with respect to one another. For example, the first sensor may include indicia relating to the second sensor having the complementary keyed interface region.

### BACKGROUND

The present disclosure relates generally to medical devices, and more particularly, to sensors for determining physiological parameters, such as plethysmographically-determined parameters and electroencephalography-derived parameters.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring certain physiological characteristics of a patient. Such devices provide doctors and other healthcare personnel with the information they need to provide the best possible healthcare for their patients. For example, photoplethysmography is a common technique for monitoring physiological characteristics of a patient, and one device based upon photoplethysmography techniques is commonly referred to as a pulse oximeter. Pulse oximeters may be used to measure and monitor various blood flow characteristics of a patient. A pulse oximeter may be utilized to monitor the blood oxygen saturation of hemoglobin in arterial blood, the volume of individual blood pulsations supplying the tissue, and/or the rate of blood pulsations corresponding to each heartbeat of a patient. In fact, the “pulse” in pulse oximetry refers to the time-varying amount of arterial blood in the tissue during each cardiac cycle.

A patient in a hospital setting may be monitored by a variety of medical devices, including devices based on pulse oximetry techniques. For example, a patient may be monitored with a pulse oximetry device, which may be appropriate for a wide variety of patients. Depending on the patient's clinical condition, a physician may also monitor a patient with a regional saturation monitor placed on the patient's head to determine if the patient is at risk of hypoxia. If a patient is scheduled for surgery, additional monitoring devices may be applied. One such device may include a sensor for bispectral index (BIS) monitoring to measure the level of consciousness by algorithmic analysis of a patient's electroencephalograph during general anesthesia. Examples of parameters assessed during the BIS monitoring may include the effects of anesthetics, evaluating asymmetric activity between the left and right hemispheres of the brain in order to detect cerebral ischemia, and detecting burst suppression. Such monitoring may be used to determine if the patient's anesthesia level is appropriate and to maintain a desired anesthesia depth.

Proper medical sensor placement may be complex if multiple sensors (e.g., pulse oximetry and regional saturation sensors) are used on the patient's tissue at the same time. In particular, the sensors may physically interfere with one another. For example, certain types of sensors may be configured for a particular geometric configuration (e.g., to be placed at a particular location) on the patient's tissue. While these locations may be different, the bulk of the sensors (e.g., due to their respective cables and/or sizes) may interfere with the ability of the sensors to be appropriately positioned.

Additionally, during BIS monitoring, multiple electrodes are applied directly to a patient's skin to acquire the EEG signal. Because BIS monitoring sensors are applied for patient monitoring during specific medical procedures, preexisting medical sensors (e.g., pulse oximetry sensors) may already be in place and may occupy a preferred BIS sensor location on the patient's tissue. While the sensors may be repositioned to accommodate the BIS sensor, such repositioning may affect the adhesion of the sensor to the skin.

### BRIEF DESCRIPTION OF THE DRAWINGS

Advantages of the disclosed techniques may become apparent upon reading the following detailed description and upon reference to the drawings in which:

FIG. 1 is a perspective view of a monitoring system configured to be used with a pulse oximetry sensor and a regional oximetry sensor in accordance with an embodiment;

FIG. 2 is a front view of a monitoring system configured to be used with a BIS sensor in accordance with an embodiment;

FIG. 3 is a top view of a sensor arrangement including sensors having keyed interface regions in accordance with an embodiment;

FIG. 4 is a top view of a sensor arrangement including sensors having keyed interface regions in accordance with an embodiment;

FIG. 5 is a top view of a sensor arrangement including sensors having keyed interface regions in accordance with an embodiment;

FIG. 6 is perspective view of a sensor kit including sensors having keyed interface regions that may be placed in a sensor arrangement in accordance with an embodiment; and

FIG. 7 is perspective view of a sensor kit including sensors having keyed interface regions that may be placed in a sensor arrangement in accordance with an embodiment.

### DETAILED DESCRIPTION OF THE SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As noted above, a patient in a medical environment may be monitored using a variety of medical devices to provide caregivers with information regarding the patient's condition. For example, a pulse oximeter may be utilized to monitor the blood oxygen saturation of hemoglobin in arterial blood and/or the rate of blood pulsations corresponding to each heartbeat of the patient. If the patient is scheduled for surgery, a sensor for BIS monitor may be utilized to monitor the patient's level of consciousness during general anesthesia. In certain situations, a regional saturation monitor may be utilized to determine if the patient is at risk of hypoxia. Unfortunately, in this example, each of these monitoring devices may include sensors that may be placed on the forehead of the patient and, thus, may physically interfere with one another. For example, as noted above, some sensors are relatively large and are configured for a particular geometric configuration on the patient's tissue, which may overlap with, or cause the bulk of the sensor to protrude into, a desired position of another sensor.

Additionally, the certain types of sensors may be configured for placement on a particular region of tissue of the patient. For example, it may be desirable to position a forehead pulse oximetry sensor above the patient's eyebrow and to center the optical components of the pulse oximetry sensor over the patient's pupil. Such proper positioning may be desirable because misplacement of a sensor may increase the algorithmic work, filtering, and artifacting to obtain the physiological characteristics from the physiological signal of the sensor. Unfortunately, caregivers may experience difficulty in determining a desired placement of a sensor and/or in positioning a sensor in a desired position when multiple sensors are applied to the patient.

Accordingly, the present disclosure is generally directed to designs or shapes for sensors that include features to facilitate proper placement of the sensors on the patient's tissue and proper placement of the sensors with respect to other sensors applied to the patient. For example, such sensors may include pulse oximetry sensors, regional oximetry sensors, BIS sensors, or other medical sensors configured to measure any suitable physiological characteristic. Each sensor may include a keyed interface region (e.g., a keyed edge or a mating edge) that is configured to align with (e.g., fit together with, mate with, or interlock) a complementary keyed interface region (e.g., a receiving keyed interface region) of another sensor. As defined herein, a complementary keyed interface region is a portion of a sensor that only aligns with a keyed interface region of another sensor in one particular spatial relationship. For example, the keyed interface region may include at least one geometric feature complementary to another geometric feature of a separate and distinct sensor. The at least one geometric feature may include, for example, a protrusion, a groove, a curved portion, a tab, a notch, a cutout, or the like. Additionally, the sensors may include features to facilitate proper placement of the sensor on the patient's tissue in relation to the anatomical features of the patient (e.g., an eyebrow of the patient) and other sensors that may be applied to the patient's tissue. For example, the sensors may be provided with text, arrows, color-coding, alignment lines, and/or any other suitable features.

With the foregoing in mind, FIG. 1 illustrates an embodiment of a patient monitoring system 10 including a monitor 12 that may be used in conjunction with a pulse oximetry sensor 14 and/or a regional oximetry sensor 16. As will be described in more detail below, the pulse oximetry sensor 14 and/or the regional oximetry sensor 16 may be configured to align with (e.g., fit into) one or more additional sensors 18 (e.g., electroencephalography (EEG) sensors, BIS sensors, pulse oximetry sensors, regional oximetry sensors, etc.). Although the illustrated embodiment of the patient monitoring system 10 relates to photoplethysmography, the patient monitoring system 10 may be configured to obtain a variety of medical measurements using a suitable medical sensor. Additionally, it should be noted that, in certain embodiments, the pulse oximetry sensor 14 and the regional oximetry sensor 16 may be communicatively coupled to separate monitors (e.g., the monitor 12 and a second monitor). Furthermore, while the illustrated embodiment of the pulse oximetry sensor 14 and the regional oximetry sensor 16 relate to sensors for use on a patient's forehead, it should be understood that, in certain embodiments, the features of the pulse oximetry sensor 14 and/or the features of the regional oximetry sensor 16 may be incorporated into medical sensors for use on other tissue locations, such as the temple, the finger, the toes, the heel, the ear, or any other appropriate measurement site.

The pulse oximetry sensor 14 may include one or more emitters 20 and one or more detectors 22 to obtain a physiological signal of a blood perfused tissue region of the patient. Generally, the light passed through the tissue is selected to be of one or more wavelengths that are absorbed by the blood in an amount representative of the amount of the blood constituent present in the blood. The amount of light passed through the tissue varies in accordance with the changing amount of blood constituent and the related light absorption. For example, the light from the emitter 20 may be used to measure blood oxygen saturation, water fractions, hematocrit, or other physiological parameters of the patient. In certain embodiments, the emitter 20 may emit at least two (e.g., red and infrared) wavelengths of light. However, any appropriate wavelength (e.g., green, yellow, etc.) and/or any number of wavelengths (e.g., three or more) may be used. The pulse oximetry sensor 14 may include a sensor body 24 to house the emitter 20, the detector 22, and the associated circuitry. The pulse oximetry sensor 14 may be communicatively coupled to the monitor 12 via a cable 26 and a plug 28 connected to a sensor port at the monitor 12. However, in other embodiments, the pulse oximetry sensor 14 may be configured to establish a wireless communication with the monitor 12 using any suitable wireless standard. By way of example, the pulse oximetry sensor 14 may be capable of communicating using one or more of the ZigBee standard, WirelessHART standard, Bluetooth standard, IEEE 802.11x standards, or MiWi standard.

While the pulse oximetry sensor 14 may generate a physiologic signal that is representative of the patient's systemic arterial oxygen saturation, the regional oximetry sensor 16 may obtain a physiological signal representative of the blood oxygen saturation within the venous, arterial, and capillary systems within an interrogated tissue region of the patient. In certain embodiments, the interrogated region of patient tissue may include a particular location in the brain, the abdomen, the kidney, the liver, and/or any other suitable location. The regional oximetry sensor 16 may include one or more emitters 30 and one or more detectors 32 to obtain the physiologic signal of the interrogated region. As illustrated, the regional oximetry sensor 16 may include two emitters 30 (e.g., for emitting two wavelengths of light) and two detectors 32, with one detector 32 relatively “close” to the two emitters 30 and one detector 32 relatively “far” from the two emitters 30. Light intensity of multiple wavelengths may be received at both the “close” and the “far” detectors 32. For example, if two wavelengths are used, the two wavelengths may be contrasted at each location and the resulting signals may be contrasted to arrive at a regional saturation value that pertains to additional tissue through which the light received at the “far” detector passed (tissue in addition to the tissue through which the light received by the “close” detector passed, e.g., the brain tissue), when it was transmitted through a region of a patient (e.g., a patient's cranium).

Surface data from the skin and skull may be subtracted out to produce a regional oxygen saturation (rSO₂) value for deeper tissues. Additionally, similar to the pulse oximetry sensor 14, the regional oximetry sensor 16 may include a sensor body 34 to house the emitters 30 and/or the detectors 32. The regional oximetry sensor 16 may be communicatively coupled to the monitor 12 via a cable 36 and a plug 38 connected to a sensor port of the monitor 12. However, in other embodiments, the regional oximetry sensor 16 may be configured to establish a wireless communication with the monitor 12 using any suitable wireless standard, such as those mentioned above.

The monitor 12 may be configured to calculate physiological characteristics relating to the physiological signal received from the pulse oximetry sensor 14 and/or physiological characteristics related to the physiological signal received from the regional oximetry sensor 16. For example, the monitor 12 may include a processor configured to calculate a patient's arterial blood oxygen saturation, the patient's pulse rate, the blood oxygen saturation of an interrogated region of tissue, and/or any other suitable physiological characteristics. In certain embodiments, the processor of the monitor 12 may be configured to read and execute coded instructions stored in a memory of the monitor 12. The monitor 12 may also include a display 40 to display physiological characteristics, historical trends of physiological characteristics, other information about the system (e.g., instructions for placement of the pulse oximetry sensor 14 and/or the regional oximetry sensor 16), and/or alarm indications. The monitor 12 may include various input components 42, such as knobs, switches, keys and keypads, buttons, etc., to provide for operation and configuration of the monitor 12. Additionally, the monitor 12 may include various circuitry (e.g., drive circuitry, amplifiers, filters, A/D converters) to control the operation of the pulse oximetry sensor 14 and/or the regional oximetry sensor 16.

The monitor 12 may be any suitable monitor, such as a pulse oximetry monitor available from Covidien LP. Furthermore, to upgrade conventional operation provided by the monitor 12 to provide additional functions, the monitor 12 may be coupled to a multi-parameter patient monitor 44 via a cable 46 connected to a sensor input port or via a cable 48 connected to a digital communication port. In addition to the monitor 12, or alternatively, the multi-parameter patient monitor 44 may be configured to calculate physiological parameters and to provide a central display 50 for the visualization of information from the monitor 12 and from other medical monitoring devices or systems. The multi-parameter monitor 44 includes a processor that may be configured to execute code. The a multi-parameter monitor 44 may also include various input components 52, such as knobs, switches, keys and keypads, buttons, etc., to provide for operation and configuration of the a multi-parameter monitor 44. In addition, the monitor 12 and/or the multi-parameter monitor 44 may be connected to a network to enable the sharing of information with servers or other workstations.

As noted above, the pulse oximetry sensor 14 and/or the regional oximetry sensor 16 may be configured to align with (e.g., fit into) one or more sensors 18. As noted above, in accordance with present embodiments, the sensor body 24 of the pulse oximetry sensor 14 and/or the sensor body 34 of the regional oximetry sensor 16 may be fabricated to include at least one keyed interface region 54 to align with a complementary keyed interface region 56 of another sensor 18. Various embodiments of such keyed interface regions will be described in more detail below with respect to FIGS. 3-5. The sensor body 24 of the pulse oximetry sensor 14 and/or the sensor body 34 of the regional oximetry sensor 16 may be formed from any suitable material, including rigid or conformable materials, such as fabric, paper, rubber, or elastomeric compositions (including acrylic elastomers, polyimide, silicone rubber, celluloid, PMDS elastomer, polyurethane, polypropylene, acrylics, nitrile, PVC films, acetates, and latex) and may be shaped during fabrication or post-fabrication (e.g., by cutting). Furthermore, as will be described in more detail below with respect to FIGS. 3-5, the sensor bodies 24 and 34 may be include various identifying features (e.g., indicia, alignment lines, and/or color-coding) to facilitate the positioning of the pulse oximetry sensor 14 and the regional oximetry sensor 16 on the patient's tissue and with respect to other sensors 18.

While certain disclosed embodiments include keyed sensors with optical elements configured for pulse oximetry or regional oximetry measurements, it is also contemplated that other medical sensors, such as EEG sensors and/or BIS sensors, may also include the keyed interface 54. In particular, BIS sensors are often applied to a patient's forehead during surgical procedures. However, other sensors (e.g., the pulse oximetry sensor 14 and/or the regional oximetry sensor 16) may already be in place on the patient's forehead. Because BIS sensors have multiple electrodes that are designed to be placed in particular locations on the forehead, the presence of other sensors on the forehead may interfere with proper positioning of the BIS sensor. By providing BIS sensors, pulse oximetry sensors 14, regional oximetry sensor 16, and/or other medical sensors with shapes (e.g., the keyed interface 54) and features (e.g., indicia, alignment lines, and/or color-coding), the sensors provided herein may facilitate the proper positioning of BIS sensors.

With the foregoing in mind, FIG. 2 is a front view of an embodiment of a patient monitoring system 80 including an electroencephalography (EEG) monitor 82 that may be used in conjunction with a BIS sensor 84. As will be described in more detail below, the BIS sensor 84 may be configured to align with (e.g., fit into) one or more sensors 86 (e.g., the pulse oximetry sensor 14 and/or the regional oximetry sensor 16). The BIS sensor 84 includes electrodes 88 (e.g., four electrodes 88A, 88B, 88C, and 88D) that may be self adherent and self prepping to temple and forehead areas of a patient. The electrodes 88 are used to acquire EEG signals. The BIS sensor 84 may include a paddle connector 90, which couples through a connector 92 to a cable 94 (e.g., a patient interface cable), which in turn may be coupled through a connector 95 to a cable 96 (e.g., a pigtail cable). In certain embodiments, the BIS sensor 84 may be coupled to the cable 96 thereby eliminating the cable 94. The cable 96 may be coupled to a digital signal converter 98, which in turn is coupled to a cable 100 (e.g., a monitor interface cable). In certain embodiments, the digital signal converter 98 may be embedded in the EEG monitor 82 to eliminate the cables 96 and 100. Cable 100 may be coupled to the EEG monitor 82 via a port 102 (e.g., a digital signal converter port).

The EEG monitor 82 may be capable of calculating physiological characteristics relating to the EEG signal received from the BIS sensor 84. For example, the EEG monitor 82 may be capable of algorithmically calculating BIS from the EEG signal. BIS is a measure of a patient's level of consciousness during general anesthesia. Further, the EEG monitor 82 may include a display 104 capable of displaying physiological characteristics, historical trends of physiological characteristics, other information about the system (e.g., instructions for placement of the BIS sensor 84 on the patient), and/or alarm indications. For example, the EEG monitor 82 may display a patient's BIS value 108. The BIS value 108 represents a dimensionless number (e.g., ranging from 0, i.e., silence, to 100, i.e., fully awake and alert) output from a multivariate discriminate analysis that quantifies the overall bispectral properties (e.g., frequency, power, and phase) of the EEG signal. For example, a BIS value 108 between 40 and 60 may indicate an appropriate level for general anesthesia. The EEG monitor 82 may also display a signal quality index (SQI) bar graph 110 (e.g., ranging from 0 to 100) which measures the signal quality of the EEG channel source(s) based on impedance data, artifacts, and other variables. The EEG monitor 82 may also display an electromyograph (EMG) bar graph 112 (e.g., ranging from 30 to 55 decibels) which indicates the power (e.g., in decibels) in the frequency range of 70 to 110 Hz. The frequency range may include power from muscle activity and other high-frequency artifacts. The EEG monitor 82 may further display a suppression ratio (SR) 114 (e.g., ranging from 0 to 100 percent), which represents the percentage of epochs over a given time period (e.g., the past 63 seconds) in which the EEG signal is considered suppressed (e.g., low activity). In certain embodiments, the EEG monitor 82 may also display a burst count for the number of EEG bursts per minute, where a “burst” is defined as a short period of EEG activity preceded and followed by periods of inactivity or suppression. The EEG monitor 82 may also display the EEG waveform 116. In certain embodiments, the EEG waveform 116 may be filtered. The EEG monitor 82 may also display trends 118 over a certain time period (e.g., one hour) for EEG, SR, EMG, SQI, and/or other parameters. In certain embodiments, the EEG monitor 82 may display stepwise instructions for placing the BIS sensor 84 on the patient. In addition, the EEG monitor 82 may display a verification screen verifying the proper placement of each electrode 88 of the BIS sensor 84 on the patient. In certain embodiments, the EEG monitor 82 may store instructions on a memory specific to a specific sensor type or model. In other embodiments, the BIS sensor 84 may include a memory that provides the instructions to the EEG monitor 82.

Additionally, the EEG monitor 82 may include various activation mechanisms 120 (e.g., buttons and switches) to facilitate management and operation of the EEG monitor 82. For example, the EEG monitor 82 may include function keys (e.g., keys with varying functions), a power switch, adjustment buttons, an alarm silence button, and so forth. It should be noted that in other embodiments, the parameters described above and the activation mechanisms 120 may be arranged on different parts of the EEG monitor 82. In other words, the parameters and activation mechanisms 120 need not be located on a front panel 122 of the EEG monitor 82. Indeed, in some embodiments, activation mechanisms 120 are virtual representations in a display or actual components disposed on separate devices. In addition, the activation mechanisms 120 may allow selecting or inputting of a specific sensor type or model in order to access instructions stored within the memory of the BIS sensor 84.

The BIS sensor 84 may include a sensor body 124, which may function as the structural support for the electrodes 88. The sensor body 124 may include one or more layers (e.g., a base structural layer, an adhesive layer, and/or a foam layer). The sensor body 124 may be formed from any suitable material, including rigid or conformable materials, such as flexible polymeric materials (e.g., polyester, polyurethane, polypropylene, polyethylene, polyvinylchloride, acrylics, nitrile, PVC films, and acetates), foam materials (e.g., polyester foam, polyethylene foam, polyurethane foam, or the like), and adhesives (e.g., an acrylic-based adhesive, a supported transfer tape, an unsupported transfer tape, or any combination thereof). Additionally, similar to the sensor body 24 of the pulse oximetry sensor 14 and/or the sensor body 34 of the regional oximetry sensor 16 as described above with respect to FIG. 1, the sensor body 124 of the BIS sensor 84 may include the keyed interface region 54 to align with the complementary keyed interface region 56 of the additional sensor 86 (e.g., the pulse oximetry sensor 14, the regional oximetry sensor 16, and/or an additional BIS sensor 84). Furthermore, as will be described in more detail below with respect to FIGS. 3-5, the sensor body 124 may be formed to include various features (e.g., indicia, alignment lines, and/or color-coding) to facilitate the positioning of BIS sensor 84 on the patient's tissue and with respect to other sensors 86.

For example, FIG. 3 illustrates an embodiment of a sensor arrangement 138 including various medical sensors having shapes and features to facilitate proper placement of the sensors on a patient's tissue and proper placement of the sensors with respect to one another. Specifically, the sensor arrangement 138 includes a first sensor 140 (e.g., the pulse oximetry sensor 14), a second sensor 142 (e.g., the BIS sensor 84), a third sensor 144 (e.g., the regional oximetry sensor 16), and a fourth sensor 146 (e.g., the regional oximetry sensor 16) applied to the forehead 148 of a patient. However, it should be noted that any number of sensors may be applied to the patient (e.g., one or more) and any type of sensors may be used. Additionally, each sensor may include a respective sensor body (e.g., a first sensor body 152, a second sensor body 154, a third sensor body 156, and a fourth sensor body 158) and a respective cable (e.g., a first cable 160, a second cable 162, a third cable 164, and a fourth cable 166). However, it should be noted that, in certain embodiments, one or more of the sensors 140, 142, 144, and 146 may share a cable.

As noted above, the sensors as described herein may each include an embodiment of the keyed interface region 54 to facilitate the placement of each sensor. For example, the first sensor body 152 may include a keyed interface region 168 to align with a keyed interface region 170 of the second sensor body 154. The first sensor body 152 may also include a keyed interface region 172 to align with a keyed interface region 174 of the third sensor body 156. Similarly, the second sensor body 154 may include a keyed interface region 176 to align with a keyed interface region 178 of the fourth sensor body 158. However, it should be noted that in other embodiments, the sensors 140, 142, 144, and 146 may be configured to align with other sensors and may have any suitable shape/geometry that facilitates placement in the manner described herein. By way of example, the keyed interface region 168 of the first sensor body 152 may be configured to align with the keyed interface region 178 of the fourth sensor body 158. Furthermore, the sensors 140, 142, 144, and 146 may include additional keyed interface regions to facilitate the placement of additional sensors, or to accommodate other devices or device periphery (e.g., sensor cables, bandages).

Generally, the keyed interface regions 168, 170, 172, 174, 176, and 178 provide at least one geometric feature that corresponds with a geometric feature of a complementary (i.e., “matching”) keyed interface region. For example, the geometric feature may include a protrusion, a groove, a curved portion, a tab, a notch, or the like. Accordingly, a geometric feature such as a protrusion may be configured to align with a geometric feature such as a groove. In certain embodiments, each “pair” of keyed interface regions (e.g., the keyed interface regions 168 and 170, the keyed interface regions 172 and 174, and the keyed interface regions 176 and 178) may include a unique geometric feature. In this manner, a caregiver may more readily identify which sensors are configured to be placed adjacent to one another and thus, may more readily position the sensors 140, 142, 144, and 146 in the sensor arrangement 138. Further, the keyed interface regions 168 and 172 may facilitate proper placement of the sensors 142, 144, and 146 after the first sensor 140 is positioned on the patient. That is, the positioning of the first sensor 140 and the keyed interface regions 168 and 172 of the first sensor 140 may provide information to the caregiver regarding the proper positioning of additional sensors (e.g., the second sensor 142 and/or the third sensor 144) relative to the first sensor 140. This may enable the caregiver to more readily arrange the sensors 140, 142, 144, and 146, which may save time. Additionally, the geometry of the keyed interface regions 168, 170, 172, 174, 176, and 178 may be selected to accommodate the positioning of the sensors 140, 142, 144, and 146 in the sensor arrangement 138. That is, certain sensors may be relatively large and may physically interfere with a desired placement of other sensors. Accordingly, in certain embodiments, the geometry of the keyed interface regions 168, 170, 172, 174, 176, and 178 may be selected to decrease the surface area of the respective sensor 140, 142, 144, and 146 to facilitate the positioning of other sensors.

The sensors 140, 142, 144, and 146 may include additional features or indicia to further facilitate the placement of the sensors 140, 142, 144, and 146. In certain embodiments, the sensors 140, 142, 144, and 146 may include one or more labels 180 relating to the placement of the sensors 140, 142, 144, and 146 with respect to anatomical features of the patient. The labels 180 may include one or more markings (e.g., arrows, lines, symbols, and/or shapes) and text to help a caregiver identify a desired position and orientation of each sensor 140, 142, 144, and 146. As used herein, a desired (e.g., preferred) position and orientation of a sensor is a placement of the sensor on a patient which results in accurate and reproducible measurements. The labels 180 may identify any suitable anatomical feature of the patient, such as an eyebrow, the hairline, an eye, a pupil, an ear, the nose, a finger, a toe, the bellybutton, the collarbone, a nipple, or the like. Additionally, for certain anatomical features, the label 180 may indicate right or left (e.g., left eyebrow). For example, it may be desirable to position a pulse oximetry sensor above an eyebrow of the patient. Accordingly, in embodiments in which the first sensor 140 is the pulse oximetry sensor 14, the label 180 of the first sensor 140 may include arrows and the word “eyebrow.” Similarly, it may be desirable to position a portion of a BIS sensor (e.g., the second and/or third electrode) over an eyebrow of the patient. Accordingly, in embodiments in which the second sensor 142 is the BIS sensor 84, the label 180 of the second sensor 142 may include arrows and the word “eyebrow.”

Additionally or alternatively, the sensors 140, 142, 144, and 146 may include one or more labels 182 relating to the placement of the sensors 140, 142, 144, and 146 relative to their respective neighboring sensors. The labels 182 may include markings (e.g., arrows, lines, symbols, and/or shapes), text, and/or numerical values to help a caregiver identify a desired position and orientation of each sensor 140, 142, 144, and 146. Specifically, the labels 182 may be located near a keyed interface region and may identify the sensor having the complementary keyed interface region. For example, the first sensor 140 may include a label 182 proximate to the keyed interface region 168, which may include text identifying the second sensor 142 (i.e., Sensor 2). It should be noted, however, that the text may additionally or alternatively identify a sensor by type (e.g., an EEG sensor, a pulse oximetry sensor, a regional oximetry sensor) and/or by a brand name (e.g., BIS, MAXFAST™, or INVOS®). Additionally, in certain embodiments, each sensor 140, 142, 144, and 146 may include a label (not shown) identifying the sensor. For example, the sensors 140, 142, 144, and 146 may include text and/or graphical indicia identifying the sensor by number (e.g., Sensor 1), by type (e.g., pulse oximetry sensor), and/or by brand (e.g., MAXFASTT™).

As noted above, certain sensors may be relatively large and may physically interfere with a desired placement of other sensors. Similarly, the cables of certain sensors may physically interfere with a desired placement of other sensors. By way of example, the BIS sensor 84 includes a relatively large cable (e.g., the paddle connector 90, the connector 92, and/or the cable 94). Furthermore, because of the positioning of the BIS sensor 84, the cable of the BIS sensor 84 may be proximate to the center of the forehead 148 and, thus, may interfere with the positioning of other sensors on the forehead 148. Thus, in certain embodiments, one of the sensors on the forehead may include an indentation to accommodate the cable of the BIS sensor 84 and/or the cable of the BIS sensor 84 may be repositioned to minimize the interference with the placement of the other sensors.

For example, FIG. 4 illustrates a sensor arrangement 200 including the sensors 140, 142, 144, and 146 and at least one repositioned sensor cable. In particular, in the illustrated embodiment, the second cable 162 of the second sensor 142 is repositioned such that the second cable 162 may fit in an open space between the third and fourth sensors 144 and 146. It should be noted that FIG. 4 illustrates one embodiment, and the second cable 162 and/or any other sensor cables may be repositioned on their respective sensors to any suitable location. By way of example, in certain embodiments, the sensor cables 160, 162, 164, and/or 166 may be repositioned such that all of the sensor cables 160, 162, 164, and 166 extend in one direction (e.g., to tie the sensor cables 160, 162, 164, and 166 together), two directions, or the like. Additionally, FIG. 4 illustrates other embodiments of the keyed interface regions 168, 170, 172, 174, 176, and 178. In particular, the keyed interface regions 172, 174, 176, and 178 include combinations of grooves and protrusions. It should be noted that the keyed interface regions 172, 174, 176, and 178 may include any suitable number of grooves and/or protrusions. For example, the keyed interface regions 168 and 170 include two curved protrusions.

While the labels 180 and 182 and the keyed interface regions 168, 170, 172, 174, 176, and 178 provide information to a caregiver relating to the positioning of the sensors 140, 142, 144, and 146, in certain embodiments, it may be desirable to provide additional features to further facilitate the positioning of the sensors 140, 142, 144, and 146. Accordingly, FIG. 5 illustrates a sensor arrangement 220 including the sensors 140, 142, 144, and 146 having various features to facilitate positioning. For example, the sensors 140, 142, 144, and 146 may include regions that are color-coded and/or include other markings (e.g., shading, cross-hatching, line quality, indicia). More specifically, the “pairs” of the keyed interface regions 168, 170, 172, 174, 176, and 178 may include such color-coding and/or markings. For example, the keyed interface regions 168 and 170 may include a first color 222 (e.g., blue), the keyed interface regions 172 and 174 may include a second color 224 (e.g., yellow), and the keyed interface regions 176 and 178 may include a third color 226 (e.g., red). It should be noted that any suitable color or any suitable combination of colors may be used. The color-coding and/or other markings may be advantageous to further facilitate a caregiver in identifying the complementary (i.e., paired) keyed interface regions. Furthermore, while the labels 182 indicating the neighboring sensor are not illustrated in FIG. 5, in certain embodiments, the labels 182 may be included along with the first, second, and third colors 222, 224, and 226.

Additionally or alternatively, the sensors 140, 142, 144, and 146 may include regions that are color-coded and/or include other markings (e.g., shading, cross-hatching, line quality, indicia) to indicate directionality of the sensors 140, 142, 144, and 146 and/or proximity to an anatomical feature (e.g., an eyebrow or the hairline). For example, in the sensor arrangement 220, the first and second sensors 140 and 142 are positioned on the lower half of the forehead 148, and the third and fourth sensors 144 and 146 are positioned above the first and second sensors 140 and 142 on the upper half of the forehead 148. Thus, in certain embodiments, the first and second sensors 140 and 142 may each include a region 228 having a color (e.g., a fourth color 230) and/or other markings to indicate that the first and second sensors 140 and 142 are intended to be positioned with the regions 228 proximate to the eyebrows of the patient. Similarly, the third and fourth sensors 144 and 146 may each include a region 232 having a color (e.g., a fifth color 234) and/or other markings to indicate that the sensors 144 and 146 are intended to be positioned with the regions 232 proximate to the hairline (i.e., the top of the forehead 148) of the patient. In other embodiments, the regions 228 and/or 232 may indicate directionality. By way of example, the region 232 may indicate the top of the sensor 144 (i.e., proximate to the patient's hairline) when the sensor 144 is applied to the patient. Additionally, the labels 180 (e.g., text corresponding to an anatomical feature) may be included along with the colors 230 and 232.

As noted above, certain types of sensors may be configured for placement on a particular region of tissue of the patient. Misplacement of a sensor (i.e., the sensing components of the sensor) may increase the algorithmic work, filtering, and artifacting to obtain the physiological characteristics from the physiological signal of the sensor. In certain cases, an appropriate signal may not be obtained due to the misplacement of the sensor. Thus, in certain embodiments, the sensors 140, 142, 144, and 146 may include alignment features (e.g., lines, arrows, circles, squares, etc.) to facilitate positioning of the sensing components of the sensors 140, 142, 144, and 146. For example, it may be desirable to position the optical components of a forehead pulse oximetry sensor over the patient's pupil. Thus, in embodiments in which the first sensor 140 is the pulse oximetry sensor 14, the first sensor 140 may include alignment lines 238. When the alignment lines 238 are aligned with a vertical axis 240 of the patient's pupil, the optical components (e.g., the emitter 20 and the detector 22) of the sensor 140 may be placed in a desired position, such as a position suitable for obtaining accurate measurements. Similarly, it may be desirable to position the sensing components (e.g., the electrodes) of a BIS sensor in particular tissue regions. Accordingly, in embodiments in which the second sensor 142 is the BIS sensor 84, the second sensor 142 may include an alignment feature 242. For example, the alignment feature 242 may include a circle and/or number designating the electrode (e.g., the electrode 88D) and may also include some form of arrows or projections from the circle and/or number to facilitate in aligning the electrode. Specifically, the alignment feature 242 may be aligned with a vertical axis 244 that bisects the patient's face. Additionally, it may be desirable to position another electrode of a BIS sensor (e.g., the electrode 88A of the BIS sensor 84) on the patient's temple and in line with the patient's pupil. For example, in certain embodiments, the second sensor 142 may include an alignment feature 246, which may help align the electrode (e.g., the electrode 88A) with a horizontal axis 248 of the patient's pupil. It should be noted that the alignment lines 238, the alignment feature 242, and/or the alignment feature 246 may be included in other sensors (e.g., the third and fourth sensors 144 and 146) and other alignment features may be incorporated to facilitate the alignment of the sensing components of various medical sensors.

In certain embodiments, the sensors 140, 142, 144, and 146, as described above, may be packaged and provided to a medical facility for use in any suitable combination. Depending on the various sensor types, it may be desirable to package one or more of the sensors 140, 142, 144, and 146 together in a sensor kit. That is, providing a sensor kit (i.e., a combined sensor package) with one or more of the sensors 140, 142, 144, and 146 may indicate to a caregiver that the sensors 140, 142, 144, and/or 146 may be suitable to use together. Moreover, as different sensors may be suitable for different medical scenarios, it may be desirable to provide one or more sensor kits including different combinations of the sensors. For example, pulse oximetry sensors may be utilized to continuously monitor a patient's blood oxygen saturation and heart rate. Thus, it may be desirable to include pulse oximetry sensors in some or all sensor kits. BIS sensors, however, may be only be utilized while the patient is under general anesthesia (i.e., during surgery). Accordingly, BIS sensors may be included in surgical sensor kits where the patient is likely to be administered a total anesthetic. Regional oximetry sensors may be used to monitor the oxygen saturation of the patient's brain while the patient is under anesthesia (e.g., general anesthesia) and/or is undergoing a surgical procedure that may be performed in low blood flow or low blood pressure conditions. For example, regional oximetry sensors may be used during cardiopulmonary, neurological, or vascular surgical procedures. Thus, it may be desirable to include regional oximetry sensors in specialized surgical kits, such as cardiac surgery kits.

With the foregoing in mind, FIG. 6 illustrates an embodiment of a sensor kit 260 including the first sensor 140 (e.g., the pulse oximetry sensor 14) and the second sensor 142 (e.g., the BIS sensor 84). However, it should be noted that any number of sensors and/or other types of sensors may be included in the sensor kit 260. The sensor kit 260 may be a surgical sensor kit. As illustrated, the first sensor 140 may be isolated (e.g., in a separate compartment) from the second sensor 142. This may be desirable for embodiments that include the BIS sensor 84. In particular, BIS sensors 84 may include a conductive gel (e.g., silver chloride), which may, under certain conditions, corrode metals of other sensors and introduce noise into components of the other sensors. Accordingly, the sensor kit 260 may include a multi-cavity sensor tray 262 (e.g., a blister pack) having a first compartment 264 with a first removable cover 265 to contain the first sensor 140 and a second compartment 266 with a second removable cover 267, isolated from the first compartment 264, to contain the second sensor 142. The multi-cavity sensor tray 262, the first compartment 264, and/or the second compartment 266 may be formed from any suitable materials, such as a polyethylene material, a polystyrene material, a polyester material, or the like. In certain embodiments, the removable covers 265 and 267 may be configured to peel away from the first and the second compartments 264 and 266, respectively. The removable covers 265 and 267 may be formed from any suitable materials, such as a polyethylene material, a polystyrene material, a polyester material, paper, metal barrier materials (e.g., an aluminum foil material), polymeric barrier materials (e.g., biaxially oriented polyethylene terephthalate), a metalized barrier film (e.g., metalized PET), or any combination thereof. In some embodiments, the second compartment 266 may include a liner 268 (e.g., a coating). The liner 268 may include any suitable lining material that is appropriate for use in conjunction with the materials of a BIS sensor (e.g., the BIS sensor 84) and its conductive gel. For example, the liner 268 may include a silicone release material, such as a siloxane material. The liner 268 may also include one or more indentations 270 for receiving each of the electrodes of a BIS sensor (e.g., the electrodes 88 of the BIS sensor 84).

Additionally, in some embodiments, it may be desirable to provide the first compartment 264 and/or the second compartment 266 with moisture-resistant materials. Indeed, BIS sensors may be sensitive to moisture loss. Thus, in certain embodiments, it may be desirable to provide the second compartment 266 with materials to restrict moisture loss. For example, the second compartment 266 may be formed from a material having a moisture vapor transmission rate (MVTR) that is sufficiently low to reducing drying out of the BIS sensor (i.e., the conductive gel of the BIS sensor). For example, the second compartment 266 may include metal barrier materials, such as an aluminum foil material, polymeric barrier materials, such as biaxially oriented polyethylene terephthalate (BoPET), a metalized barrier film (e.g., metalized PET), or any combination thereof. Additionally, in certain embodiments, it may be desirable to provide the first compartment 264 with a moisture-resistant material. For example, certain sensors (e.g., the pulse oximetry sensor 14) may include a patient-contacting adhesive that may be sensitive to prolonged exposure to moisture. For example, a hydrocolloid adhesive may be used to provide enhanced comfort to a patient and to minimize discomfort for the patient when the sensor is removed. However, the hydrocolloid adhesive may absorb moisture and, overtime, may begin to degrade. Accordingly, to maintain the integrity of the hydrocolloid adhesive, the first compartment 264 may also include moisture-resistant materials, such as metal barrier materials (e.g., an aluminum foil material), polymeric barrier materials (e.g., biaxially oriented polyethylene terephthalate (BoPET)), a metalized barrier film (e.g., metalized PET), or any combination thereof.

FIG. 7 illustrates an embodiment of a sensor kit 280 including the first sensor 140 (e.g., the pulse oximetry sensor 14), the second sensor 142 (e.g., the BIS sensor 84), and the third sensor 144 (e.g., the regional oximetry sensor 16). However, it should be noted that any number of sensors and/or other types of sensors may be included in the sensor kit 280. For example, the sensor kit 280 may also include the fourth sensor 146 (e.g., the regional oximetry sensor 16). The sensor kit 280 may be a specialized surgical kit, such as a cardiac surgery kit. As illustrated, the sensor kit 280 may include the multi-cavity sensor tray 262 having the first and second compartments 264 and 266, as described above. The multi-cavity sensor tray 262 of the sensor kit 280 may also include a third compartment 282 with a third removable cover 284 to contain the third sensor 144. Similar to the first and second removable covers 265 and 267, the third removable cover 284 may be configured to peel away from the third compartment 282. Further, the third removable cover 288 may be formed from any suitable materials, such as a polyethylene material, a polystyrene material, a polyester material, paper, metal barrier materials (e.g., an aluminum foil material), polymeric barrier materials (e.g., biaxially oriented polyethylene terephthalate), a metalized barrier film (e.g., metalized PET), or any combination thereof. The third compartment 282 may be isolated from both the first compartment 264 and the second compartment 266, or only the second compartment 266. In other words, in certain embodiments, the first and third compartments 164 and 282, may share a common space and/or a common removable cover. Indeed, sensors of the same or similar type may be packaged together (e.g., in the same compartment) or separately (e.g., in different compartments).

The third compartment 282 may be formed from any one or a combination of suitable materials, such as a polyethylene material, a polystyrene material, a polyester material, or the like. Additionally, similar to the pulse oximetry sensor 14 as described above, regional oximetry sensors (e.g., the regional oximetry sensor 16) may include a patient-contacting adhesive, such as a hydrocolloid adhesive. Accordingly, in certain embodiments, it may be desirable to provide the third compartment 282 with materials to restrict moisture loss, such as those described above with respect to FIG. 6.

The disclosed embodiments, such as those described above for performing patient monitoring, may be interfaced to and controlled by a computer readable storage medium having stored thereon a computer program. The computer readable storage medium may include a plurality of components such as one or more of electronic components, hardware components, and/or computer software components. These components may include one or more computer readable storage media that generally store instructions such as software, firmware and/or assembly language for performing one or more portions of one or more implementations or embodiments of an algorithm as discussed herein. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, and/or solid-state or electronic memory. Other forms of non-transitory and/or tangible computer readable storage media not list may be employed with the disclosed embodiments.

A number of such components can be combined or divided in an implementation of a system. Further, such components may include a set and/or series of computer instructions written in or implemented with any of a number of programming languages, as will be appreciated by those skilled in the art. In addition, other forms of computer readable media such as a carrier wave may be employed to embody a computer data signal representing a sequence of instructions that when executed by one or more computers causes the one or more computers to perform one or more portions of one or more implementations or embodiments of a sequence.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A sensor, comprising:
one or more sensing components configured to generate a physiological signal of a patient; and
a sensor body configured to house the one or more sensing components, wherein the sensor body comprises a keyed interface region, and wherein the keyed interface region is configured to align with a complementary keyed interface region of a second sensor.

2. The sensor of claim 1, wherein the sensor comprises a pulse oximetry sensor or a regional oximetry sensor.

3. The sensor of claim 1, wherein the sensor comprises an electroencephalography sensor.

4. The sensor of claim 1, wherein the sensor body is configured to be applied to the patient's forehead.

5. The sensor of claim 1, wherein the keyed interface region comprises at least one of a protrusion or a cutout complementing a second cutout or a second protrusion, respectively, of the second sensor.

6. The sensor of claim 1, wherein the sensor body comprises one or more indicia to facilitate proper placement of the sensor on the patient.

7. The sensor of claim 6, wherein the one or more indicia comprise one or more markings indicating the proper placement of the sensor on the patient relative to an anatomical feature of the patient.

8. The sensor of claim 6, wherein the one or more indicia comprise a label identifying the second sensor and at least one arrow pointing toward the second sensor when the keyed interface region of the sensor and the complementary keyed interface region of the second sensor are aligned, and wherein the label and the at least one arrow are located on the sensor body proximate to the keyed interface region.

9. The sensor of claim 6, wherein the one or more indicia comprise a label identifying an anatomical feature of the patient and at least one arrow pointing toward the anatomical feature of the patient when the sensor body is applied to the patient.

10. A system, comprising:
a first sensor configured to generate a first physiological signal of a patient, wherein the first sensor comprises a first sensor body comprising a first keyed interface region;
a second sensor configured to generate a second physiological signal of the patient, wherein the second sensor comprises a second sensor body comprising a second keyed interface region; and
wherein the first keyed interface region is complementary to the second keyed interface region such that the first and the second keyed interface regions are configured to align when the first and the second keyed interface regions are placed alongside one another.

11. The system of claim 10, wherein the first sensor body comprises a first label identifying the second sensor and at least one arrow pointing toward the second sensor when the first and second keyed interface regions are aligned, and wherein the first label and the at least one arrow are located on the first sensor body proximate to the first keyed interface region.

12. The system of claim 10, comprising a third sensor configured to generate a third physiological signal of the patient, wherein the third sensor comprises a third sensor body comprising a third keyed interface region, and wherein the first sensor body, the second sensor body, or a combination thereof comprise a fourth keyed interface region complementary to the third keyed interface region.

13. The system of claim 12, wherein the first and second keyed interface regions comprise a first color or a first pattern of markings, and wherein the third and fourth keyed interface regions comprise a second color or a second pattern of markings.

14. The system of claim 10, wherein at least one of the first sensor body and the second sensor body comprise a label identifying an anatomical feature of the patient and at least one marking identifying a preferred placement of the first or second sensor relative to the anatomical feature when the first or the second sensor body is applied to the patient.

15. The system of claim 10, comprising a patient monitor communicatively coupled to the first sensor and comprising a processor configured to calculate a first physiological parameter of the patient based at least in part upon the first physiological signal, wherein the first sensor comprises a pulse oximetry sensor.

16. The system of claim 15, wherein the patient monitor is communicatively coupled to the second sensor and the processor is configured to calculate a second physiological parameter of the patient based at least in part upon the second physiological signal, wherein the second sensor comprises a regional oximetry sensor.

17. The system of claim 10, comprising an electroencephalography monitor communicatively coupled to the first sensor and configured to calculate a physiological parameter of the patient based at least in part upon the first physiological signal, wherein the first sensor comprises a bispectral index sensor.

18. A medical sensor kit, comprising:
a first medical sensor comprising a first sensor body, wherein the first sensor body comprises a first keyed interface region;
a second medical sensor comprising a second sensor body, wherein the second sensor body comprises a second keyed interface region complementary to the first keyed region such that the first and the second keyed interface regions are configured to align when the first and the second keyed interface regions are placed alongside one another; and
a tray comprising a first compartment configured to house the first medical sensor and a second compartment configured to house the second medical sensor, wherein the first compartment is separate from the second compartment.

19. The medical sensor kit of claim 18, wherein the first medical sensor comprises a pulse oximetry sensor and the second medical sensor comprises a bispectral index sensor, and wherein the second compartment comprises at least one of a metal barrier material, a polymeric barrier material, or a metalized barrier film.

20. The medical sensor kit of claim 19, comprising a regional oximetry sensor, wherein the regional oximetry sensor comprises a third sensor body comprising a third keyed interface region, and wherein the first sensor body, the second sensor body, or a combination thereof comprise a fourth keyed interface region complementary to the third keyed interface region, and wherein the tray comprises a third compartment configured to house the regional oximetry sensor.
